(19) United States
(12) Patent Application Publication
(10) Pub. No.: US 2007/0078290 A1
(43) Pub. Date: Apr. 5, 2007

(54) Ultrasound-based treatment methods for therapeutic treatment of skin and subcutaneous tissues

(76) Inventor: Rinat O. Esenaliev, League City, TX (US)

Correspondence Address: LOCKE LIDDELL & SAPP LLP, 600 TRAVIS, 3400 CHASE TOWER, HOUSTON, TX, 77002-3095, US

(21) Appl. No.: 11/542,057

(22) Filed: Oct. 2, 2006

Related U.S. Application Data
(60) Provisional application No. 60/722,492, filed on Sep. 30, 2005.

(51) Int. Cl. A61N 5/00

(52) U.S. Cl. 600/1

(57) ABSTRACT

The disclosure provides a method and apparatus for noninvasive and minimally-invasive treatment of skin and subcutaneous tissues with ultrasound with or without nano- or microparticles. The treatment includes, but is not limited to, hair removal, skin rejuvenation (wrinkle removal), scar removal, treatment of spider veins and varicose veins, removal of birthmarks, acne treatment, wound treatment, abnormal pigmentation and stretch mark removal, abnormal tissues in skin and subcutaneous layers, and tattoo removal. Skin and subcutaneous tissues which can be treated with the methods described include, but are not limited to, the dermis, epidermis, subcutaneous fat, connective tissue, muscle tissue, blood vessels, scar tissues, tendons, and cartilage tissues, and abnormal tissues in skin and subcutaneous layers. The disclosure is especially applicable to hair removal and skin rejuvenation.

### CROSS REFERENCE TO RELATED APPLICATIONS

This application claims priority to Provisional Patent Application No. 60/722,492, filed Sep. 30, 2005, which is incorporated herein by reference in its entirety.

### STATEMENT REGARDING FEDERALLY SPONSORED RESEARCH OR DEVELOPMENT

Not applicable.

### REFERENCE TO APPENDIX

Not applicable.

### BACKGROUND OF THE INVENTION

1. Field of the Invention

The invention relates to methods and apparatus for the non-invasive and/or minimally invasive treatment of the skin and subcutaneous layers of the skin for therapeutic applications and, more specifically, to tissue treatment methods using ultrasound waves.

2. Description of the Related Art

Treatments of the skin are widely know and used, both for therapeutic and cosmetic reasons. Therapeutically, the uses include the treatment of cancerous cells, and the treatment of burn victims. Cosmetic applications are much more numerous, and include face-lifts and neck-lifts, treatment of dyschromia (skin tone abnormalities), shrinking the skin following operations such as liposuction, abrasion of unwanted markings on the skin, and other aesthetic skin remodeling purposes. Regardless of the purpose, skin treatments are often complicated, expensive, and in some cases, largely ineffective.

A variety of dermatological techniques have been developed for a wide range of applications, with varying degrees of success. Predominant among these is the use of photothermal applications using lasers or other electromagnetic radiation generating instruments for therapeutic or cosmetic uses. For example, Altshuler, et al. [J. Phys. D: Appl. Phys., Vol. 38: pp. 2732-2747 (2005)] describe the techniques of spatial selectivity with lasers to provide an alternative approach to delivering effective and safe laser treatments. This method creates a lattice of localized areas of light-tissue interaction, termed “optical islets”, which are formed using a variety of energy sources and optical delivery systems.

Studies to produce controlled, spatially-confined thermal effects in the dermis using a 1W, 1,500 nm fiber-coupled diode laser in non-ablative dermal remodeling (NDR), or subsurfacing, was reported by Khan, et al. [Lasers Surg. Med., Vol. 36: pp. 270-280 (2005)]. According to this report, it was believed that producing numerous microscopic regions of thermal injury surrounded by uninjured dermal tissue might achieve a better combination of efficacy and safety for treatment of photoaging than other treatment modalities. As shown therein, foci of thermal injury were typically 50-150 μm in diameter, elliptical, and at controllable depths up to 550 μm.

Recently, there has been increased interest in non-ablative treatment methods involving ultrasound imaging technology. For example, Moody, et al. [Dermatol. Surg., Vol. 29 (10): pp. 997-1000 (2003)] have reported on collagen remodeling and formation after non-ablative laser irradiation, using ultrasonographic assessment methods. Using ultrasonography, it was demonstrated that an increase in dermal collagen occurred after a single treatment with a 585-nm pulsed dye laser, and that the greatest degree of neocollagenesis occurred periocularly.

Ultrasound has also been used in cervicofacial rejuvenation, using ultrasound-assisted lipectomy, allowing for the stimulation of skin retraction and for the superficial fat to be more safely accessed than can be accomplished with conventional liposection methods [see, Grotting, J. C., and Beckenstein, M. S., Plast. Reconstr. Surg., Vol. 107: pp. 847-855 (2001); Pine, J. L., et al., Plast. Surg. Nurs., Vol. 23 (3): pp. 101-108 (2003)]. While the ultrasound is used primarily for fat destruction, this technique remains an invasive technique as ultrasound probes are inserted through the skin in order to effect the destruction of tissue, and is not without complications.

Other medical applications of ultrasound technology have included enhanced gene delivery, such as delivery of factor IX plasmid DNA using ultrasound contrast agents as nucleation initiators [Miao, C. H., et al., Hum. Gene Ther., Vol. 16 (7): pp. 893-905 (2005)]; wound management such as wound cleaning, debridement, and granulation stimulation using both high frequency and low frequency ultrasound [Uhlemann, C., et al., Int. J. Low Extrem. Wounds, Vol. 2 (3): pp. 152-157 (2003)]; therapeutic application to vessel occlusion [Hwang, J. H., et al., Ultrasound Med. Biol., Vol. 31 (4): pp. 553-564 (2005)]; and, more recently, assisted drug delivery applications.

For example, in U.S. Pat. No. 6,165,440, methods and systems for enhancing drug delivery to solid tumors were described. Specifically disclosed was a method/system utilizing the interaction of electromagnetic pulses or ultrasonic radiation with nano- and microparticles for the enhancement of drug delivery in solid tumors. The particles reportedly can be attached to antibodies directed against the antigens in tumor vasculature and selectively delivered to tumor blood vessel walls. The enhancement of drug delivery in tumors by using the interactions of nanoparticles with ultrasound radiation, as well as optimal drug and gene delivery in cancer cells using ultrasound-induced cavitation, have also been reported by Larina, I. V., et al., [Technol Cancer Res. Treat., Vol. 4(2): pp. 217-226 (2005); Anticancer Res., Vol. 25(1A): pp. 149-156 (2005)] and Chumakova, O. V. et al. [Ultrasound in Med Biol., Vol. 32(5): pp. 751-758 (2006)].

A similar approach using intradermal drug delivery by low frequency sonophoresis has been described by Santoianni, et al. [Dermatology Online Journal, 10 (2): 24 (2004)], wherein the efficacy of low frequency sonophoresis (LFS) at 25 KHz produced by a sonicator apparatus for the treatment of alopecia areata, melasma, and solar lentigo was evaluated. Patients were treated with corticosteroids, followed by LFS, and evaluated over the course of several weeks, eventually illustrating that LFS enhances the penetration of topical drug agents in obtaining effects via intradermal delivery.

The use of ultrasound and ultrasonic energy in methods of hair removal has also been reported, albeit with limited successes. U.S. Pat. No. 5,989,267 to Anderson suggests a method of removing hair, involving chemically or mechanically removing the hair to expose the follicle of hair, and then treating the follicle with both ultrasound waves and photosensitizers such as aminoluvelinic acid to inhibit its ability to regenerate a hair. Removing the hair reportedly facilitates the uptake of a follicle-inactivating compound and thus allows for long-term inhibition of hair growth.

In U.S. Pat. No. 6,200,326 to Narayanan, et al., a method and apparatus for the long-term removal of a hair are suggested. According to the disclosure, ultrasonic energy is transmitted to a needle invasively passed through the skin into an individual hair follicle. The resulting cavitation of the area surrounding the hair follicle causes the hair follicle to be disrupted. The process can then be repeated for individual hair follicles over a selected region of the body.

Despite the advances in the treatment of dermatological disorders, and plastic surgery methods, there remains a need for improved treatment methods for the skin and the subcutaneous tissues thereof that are substantially non-invasive in nature. This application for patent discloses systems for use in the therapeutic treatment of dermatological and skin-related disorders using ultrasound-based treatments, as well as methods for administering such treatments.

### BRIEF SUMMARY OF THE INVENTION

The disclose provides a method and apparatus for noninvasive and minimally-invasive treatment of skin and subcutaneous tissues with ultrasound with or without the use of nano- or micro-particles. The treatment includes, but is not limited to: hair removal, skin rejuvenation, such as wrinkle removal, scars removal, treatment of spider veins and varicose veins, removal of birthmarks, abnormal pigmentation and stretch marks, and tattoo removal. Skin and subcutaneous tissues include, but are not limited to: dermis, epidermis, subcutaneous fat, connective tissue, muscle tissue, blood vessels, scar tissues, tendons and cartilage tissues.

In an aspect of the present invention, a method for the dermatological treatment of a region of skin on a mammal is described, wherein the method comprises irradiating the skin with ultrasonic radiation having a frequency ranging from about 20 kHz to about 4 GHz for a first duration of time from about 1 millisecond to about 30 minutes, and then re-exposing the skin to the ultrasonic radiation for a further duration of time at least equal to the first duration of time. In a further aspect, the method may also include applying an effective amount of cavitation-inducing nano-particles or microparticles to the region of skin.

In another aspect of the present invention, a method for removing a plurality of hair follicles from a region of skin of a mammal is described, wherein the method comprises applying a therapeutically effective amount of nanoparticles or microparticles to the surface of the skin area to be treated, and irradiating the surface of the skin with ultrasonic radiation, wherein the ultrasonic radiation disrupts the plurality of hair follicles in the region being treated.

In another aspect of the present invention, a method for rejuvenating the skin of a mammal is described, the method comprising irradiating the skin with ultrasonic radiation at a frequency suitable to penetrate to the subcutaneous tissue layer of the skin for a duration of time from about 1 millisecond to about 30 minutes, such that the ultrasonic radiation damages the subcutaneous tissue, and allows for the natural re-growth of the cellular structure.

### BRIEF DESCRIPTION OF THE SEVERAL VIEWS OF THE DRAWINGS

A more particular description, briefly summarized above, may be had by reference to the embodiments illustrated in the appended drawings, forming part of the present specification and described herein. It is to be noted, however, that the appended drawings illustrate only some embodiments described herein and are therefore not to be considered limiting of the disclosure's scope, in that there can be other equally effective embodiments.

FIG. 1 illustrates a perspective view of a section of skin having a signal generator G feeding an ultrasound transducer.

FIG. 2 illustrates a schematic diagram of an apparatus in accordance with an aspect of the present disclosure.

FIG. 3 illustrates a schematic diagram of a system for use in accordance with an aspect of the present disclosure.

FIG. 4A illustrates the cavitation signal obtained from pure water.

FIG. 4B illustrates the cavitation signal obtained from a solution of water containing PLGA nanoparticles.

FIG. 5A illustrates the cavitation activity signal in pure water over time at increased ultrasound pressures.

FIG. 5B illustrates the cavitation activity signal for a solution of water containing PLGA nanoparticles over time, at increased ultrasound pressures.

FIG. 6A illustrates the cavitation activity over time at increased pressure measured in vivo in subcutaneous tissue at a depth of 3 mm, following an injection of Optison™.

FIG. 6B illustrates the cavitation activity measured in vivo in subcutaneous tissue at a depth of 3 mm, following an injection of PLGA nanoparticles.

FIG. 7A illustrates an optical microscopy cross-sectional view of a damaged subcutaneous tissue region.

FIG. 7B illustrates an enhanced view of a damaged subcutaneous tissue region.

FIG. 8A illustrates a stained micrograph showing damage induced in pig dermal collagen using non-focused ultrasound with lipid-based microparticles.

FIG. 8B illustrates a stained micrograph showing the minor-damage induced in pig dermal collagen using non-focused ultrasound in combination with protein-based microparticles.

FIG. 8C illustrates a stained micrograph showing no induced damage in pig dermal collagen using non-focused ultrasound in combination with pure water applied topically to the surface of the dermis prior to irradiation.

FIG. 8D illustrates intact pig dermal collagen that has not been irradiated with ultrasound.

FIG. 9A illustrates a microscopic view of a hair follicle, along the hair follicle, showing the damage induced by the use of non-focused ultrasound in combination with a protein-based microparticle.

FIG. 9B illustrates a cross-sectional view of a hair follicle, showing the damage induced to the follicle by use of non-focused ultrasound in combination with a topically applied, protein-based microparticle.

FIG. 10A illustrates a stained microscopic view of the relatively minor damage induced in a hair follicle by the use of non-focused ultrasound in combination with a topically applied lipid-based microparticle.

FIG. 10B illustrates a cross-sectional microscopic view of a hair follicle exhibiting substantially no damage induced to either the hair or the hair follicle by use of non-focused ultrasound in combination with topically applied water.

While the inventions disclosed herein are susceptible to various modifications and alternative forms, only a few specific embodiments have been shown by way of example in the drawings and are described in detail below. The figures and detailed descriptions of these specific embodiments are not intended to limit the breadth or scope of the inventive concepts or the appended claims in any manner. Rather, the figures and detailed written descriptions are provided to illustrate the inventive concepts to a person of ordinary skill in the art and to enable such person to make and use the inventive concepts.

### DEFINITIONS

The following definitions are provided in order to aid those skilled in the art in understanding the detailed description of the present invention.

“Acoustic streaming”, as used herein, refers to a physical phenomenon in a liquid or liquid-like medium (including skin tissue) represented by flows of a part of the liquid medium upon irradiation by one or more ultrasonic waves.

The term “cavitation”, as used herein, refers to a physical phenomena in a medium, such as skin tissue, represented by the formation of vapor (or gas) bubbles followed by growth, oscillation, and collapse of the bubbles.

As used herein, “microconvection” refers to micron-sized displacement or other movement (flow) in at least a part of a liquid or liquid-like medium.

“Microparticle”, as used herein, refers generally to particles having a diameter from about 0.1 μm to about 9000 μm.

“Nanoparticle”, as used herein, refers generally to particles having a diameter from about 0.1 nm to about 9000 nm.

As used herein, “therapeutically effective amount” is meant to refer to the amount of therapeutic agent or other compound, such as a nanoparticle or microparticle, sufficient to elicit one or more desired effects, or achieve one or more therapeutically effective results, including hair loss, hair follicle stimulation, wrinkle reduction, scar removal, disappearance or lightening, or the like.

Similarly, as used herein, a “therapeutic effect” or “therapeutically desirable effect” refers to a change in a region being treated such that it exhibits signs of being effected in the manner desired, e.g., hair loss begins to become evident, wrinkles begin to disappear.

As used herein, “ultrasound” or “ultrasonic radiation” refers to mechanical (including acoustic or other terms of pressure) waves in a medium in the general frequency range from about 20 kHz to about 4 GHz or greater.

### DETAILED DESCRIPTION

One or more illustrative embodiments incorporating the invention disclosed herein are presented below. Not all features of an actual implementation are described or shown in this application for the sake of clarity. It is understood that in the development of an actual embodiment incorporating the present invention, numerous implementation-specific decisions must be made to achieve the developer's goals, such as compliance with system-related, business-related, government-related and other constraints, which vary by implementation and from time to time. While a developer's efforts might be complex and time-consuming, such efforts would be, nevertheless, a routine undertaking for those of ordinary skill the art having benefit of this disclosure.

In general terms, Applicants have created systems and methods for the therapeutic treatment of dermatological diseases and disorders using ultrasound-based treatments methods.

The ultrasound-based techniques for skin treatment have advantages compared to the existing techniques known and used in the dermatological arts, including laser-based techniques, chemical techniques, electrolysis, electromagnetic wave techniques, and mechanical techniques (e.g., tweezers). These techniques are not free of limitations and do not provide: permanent hair removal, substantially complete wrinkle removal (in particular, deep wrinkles), and have limitations in application to scars removal, treatment of spider veins and varicose veins, removal of birthmarks, abnormal pigmentation and stretch marks, and tattoo removal. The major limitation of the laser-based techniques (the most popular and common technique) is a limited penetration depth of the treatment dose of light in tissue (the maximum depth is about 0.5 mm) that does not allow for treatment of deep layers of the dermis, subcutaneous fat, and the other tissues. Strong light scattering and attenuation does not allow for focusing of light in desired locations in the deep tissue layers. Ultrasound can penetrate much deeper—up to several centimeters, or into the panniculus adiposus (hypodermis) layer of subcutaneous tissue. Additionally, ultrasound waves can be focused in tissues to provide damage to local areas with a desirable size and shape. This is very important for efficient skin rejuvenation because the non-damaged areas serve to provide regeneration. This is very important for hair removal as well, because the depth of location of hair follicles is several millimeters below the surface of the skin, a depth to which laser light cannot readily penetrate at the level necessary to destroy hair follicles.

The present disclosure can be used on human (or other animal) skin for the treatment of wrinkles and other changes related to photo-aging or chronologic aging (generally termed skin rejuvenation), for the treatment of diseases including skin diseases, for the reduction of acne and related disorders such as rosacea, folliculitis, pseudofolliculitis barbae or proliferative or papulosquamous disorders such as psoriasis, for treating the pancreas in diabetes, for the stimulation or reduction of hair growth, and for reduction of cellulite, warts, hypopigmentation such as port-wine stain (PWS; nevus flammeus), birthmarks, hyperhidrosis, varicose veins, pigment problems, tattoos, vitiligo, melasma, scars, stretch marks, fungal infections, bacterial infections, dermatological inflammatory disorders, musculoskeletal problems (for example, tendonitis or arthritis), to improve healing of surgical wounds, burn therapy to improve healing and/or reduce and minimize scarring, improving circulation within the skin, in vitro fertilization enhancement, and the like.

The present invention can also be useful in improving wound healing, including but not limited to chronic skin ulcers, diabetic ulcers, thermal burn injuries, viral ulcers or disorders, periodontal disease and other dental disease. The present invention may be useful in enhancing the effects of devices which create an injury or wound in the process of performing cosmetic surgery including non-ablative thermal wounding techniques for treating skin wrinkles, scars, stretch marks and other skin disorders. Under such circumstances, it may be preferable to use conventional non-ablative thermal treatments in combination with the non-thermal ultrasonic methods of the present invention. The present invention may also be used in conjunction with micro- or surface abrasion, dermabrasion, or enzymatic or chemical peeling of the skin or topical cosmeceutical applications, with or without ultrasound application to enhance treatment, as the removal of the stratum corneum (and possibly additional epithelial layers) can prove beneficial for some treatment regimen. The methods of the present invention are particularly applicable to, but are not limited to, hair removal, hair growth/hair follicle stimulation, and skin rejuvenation, as described herein.

The dermatologically therapeutic methods described herein may be formed using ultrasound irradiation alone, ultrasound irradiation in combination with nano- or microparticles, or ultrasound irradiation with a composition comprising nano- or microparticles and one or more therapeutic agents. Such ultrasound irradiation may be produced by any known ultrasound generator, and is preferably a focused ultrasound generator capable of generating and irradiating focused ultrasound waves, so as to induce cavitation in the tissues (skin and subcutaneous tissue) of a mammalian patient, such as a human. In accordance with aspects of the present disclosure, the ultrasound irradiation used in the described therapeutic methods is preferably used at a pressure ranging from about 1 bar to about 200 bar, preferably from about 10 bar to about 100 bar, more preferably at a pressure range between about 20 bar and about 60 bar, including but not limited to values within these pressure ranges, such as 27 bar and 48 bar.

A. Hair Removal

In accordance with one aspect of the present disclosure, methods for the removal of unwanted hair from a region of the body of a mammal may be accomplished using ultrasonic irradiation alone, or in combination with nano- or microparticles, which may further be optionally combined with one or more appropriate therapeutic agents. In accordance with these aspects of the present disclosure, the interaction of ultrasound irradiation with nano- or microparticles may be used for hair removal. The nano- or microparticles can be applied topically on the skin surface and penetrate into the area of the hair follicles, or may be injected directly into the skin and delivered to the specific region of the skin tissue that is desired to be effected. Irradiation of the nano- or microparticles with ultrasound can result in damage to (or complete elimination of) the hair follicles followed by reduction in hair growth or even permanent hair removal. The mechanisms of hair follicle destruction include, but are not limited to: cavitation, thermal damage, acoustic streaming, and combinations thereof. In accordance with the present disclosure, it has been found that ultrasound cavitation can be dramatically enhanced when nano- or microparticles are used in combination with the ultrasound irradiation, because they serve as cavitation nuclei. The use of nano- or microparticles substantially lowers the cavitation threshold and increases cavitation activity. Additionally, collapsing cavitation bubbles can produce strong local hydrodynamic flows that are capable of damaging hair follicles. The size of the nano- or microparticles can be in the range from about 0.1 μm to about 9000 μm (microparticle), and from about 0.1 nm to about 9000 nm (nanoparticle) and have a variety of shapes (spheres, cylinders, cubes, pyramids, rods, shells, and other shapes). The nano- and microparticles include, but are not limited to gas particles (bubbles), liquid particles, solid (polymer, metal, dielectric, semiconductor) particles, lipid-coated or their combination or composites. Preferably, they contain a gas that substantially lowers cavitation threshold and increase cavitation activity. Preferably, in accordance with one aspect of the present disclosure, the nanoparticles and microparticles are biodegradable including, but not limited to nano- or microbubbles, and porous and non-porous solid particles. Examples of suitable nano- or microbubbles include, but are not limited to, ultrasound contrast agents such as ALBUNEX®, OPTISON™ (Amersham, Mallinkrodt; consists of heat denatured human albumin microcapsules containing the gas octafluoropropane, wherein each mL of microsphere suspension contains 5-8×10⁸ microspheres with a mean diameter in the 2-4.5 micron size range and 220 μg octafluoropropane), DEFINITY® (Bristol Myers Medical Imaging), IMAGENT™ (Photogen Inc.; a product that consists of lipid microspheres containing pefluorohexane where the lipid shell is comprised of the phospholipid DMPC, and each mL of suspension contains approximately 1.4×10⁹ microparticles having a mean diameter less than 3 microns and 92 μg of perfluorohexane), Levovist®, and bioSphere™ (Point Biomedical, San Carlos, Calif.), as well as other microparticles known in the art to be suitable for use as ultrasound contrast agents, including those described and referenced in U.S. Patent Publication No. 2005/0271591 A1, incorporated herein by reference in its entirety. The porous (gas filled) polymer nano- or microparticles include, but are not limited to those nano- and microparticles which are biocompatible and/or biodegradable, including poly(lactic acid) (PLA), poly(glycolic acid) (PGA), and poly(lactic-co-glycolic acid) (PLGA) particles, as well as similarly biocompatible nano- and microparticles. Solid (porous and non-porous) particles can produce more stable cavitation sufficient to destroy hair follicles compared to that produced by gas particles.

In accordance with one aspect of the present disclosure, the ultrasound-based hair removal methods can be performed without nano- or microparticles. However, it has been found that the use of the nano- or microparticles is preferable because they allow for efficient hair removal at low (diagnostic, close to diagnostic, or physiotherapeutic) levels of ultrasound irradiation.

The frequency of the ultrasound irradiation suitable for use with the therapeutic methods described herein can range from about 20 kHz to about 900 MHz, more preferably from about 20 kHz to about 5 MHz, more preferably from about 20 kHz to about 1 MHz, and more preferably from about 20 kHz to about 500 kHz, including ranges and values between these ranges, such as between about 21 kHz and about 100 kHz, and between about 55 kHz and about 95 kHz. In one embodiment, the frequency of ultrasound irradiation suitable for use ranges from about 20 kHz to about 100 MHz. Preferably, the frequency is close to resonant frequency of the nano- or microparticles (MHz range) or hair follicles (kHz range). Two or more frequencies can be used for more efficient hair removal. Other ultrasound parameters (duration of irradiation, duty cycle, power, intensity, irradiation spot, etc.) can be selected to provide best treatment effect at minimal damage to other tissues.

The ultrasound-based hair removal (with or without nano- or microparticles) can be performed in combination with other hair removal techniques including, but not limited to laser- (or in general, light-) based techniques, chemical techniques (depilatories, etc.), electrolysis, mechanical removal (mechanical epilators, tweezers, etc.). To provide permanent effect, the ultrasound-based hair removal (follicle destruction) can be used after laser hair removal because the nano- or microparticles can easily penetrate through the channel obtained after laser hair removal.

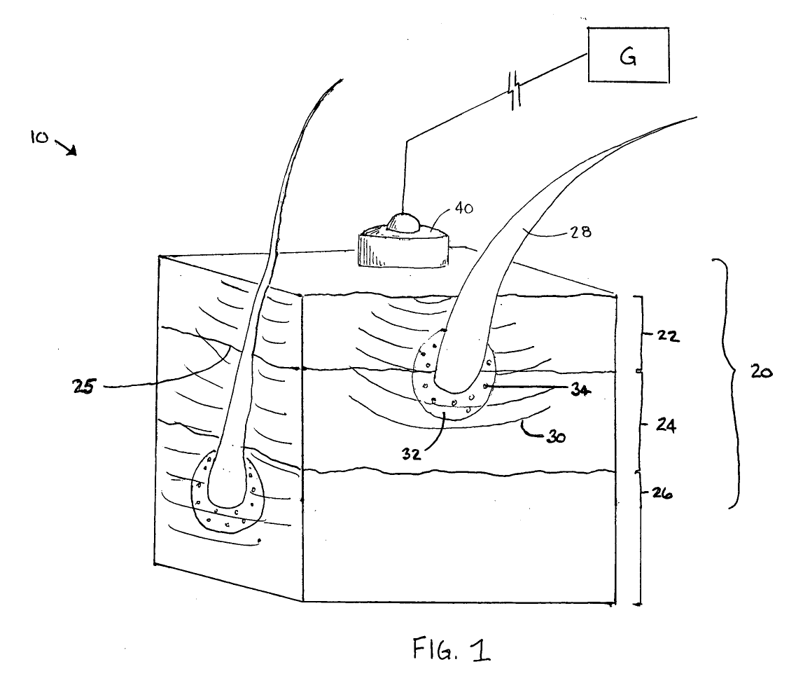

The application of ultrasound-based hair removal, with the use of nanoparticles, is illustrated schematically in FIG. 1. The application involves a region of skin 10, shown in a perspective view illustrating the various subcutaneous layers 20 of the skin. Specifically, the skin is divided into the epidermis 22, the dermis 24, and the hypodermis 26 (also known as the subcutaneous tissue, or panniculus adiposus). The epidermis extends from about 0.05 mm to about 1.5 mm below the surface of the skin, and comprises the lamina lucinda and the lamina densa. The dermis 24 is below the epidermis, is separated from the epidermis by the dermoepidermal junction 25, and has a thickness from about 1.4 mm to about 4 mm, depending upon the location. The dermis comprises the upper, papillary dermis and the lower, reticular dermis (known as the “deep layer”). Below the dermis is the subcutaneous tissue 26, which has an average thickness of from about 1 cm to about 4 cm, although this thickness increases on obese individuals.

As further illustrated in FIG. 1, a hair follicle 28 extends into the dermis layer 24. An ultrasound transducer 40, operably connected to a signal generator G, can be placed onto the surface of the skin and cause the generation of ultrasound waves 30, which depending upon their frequency penetrate to the desired depth below the skin. In other embodiments, the ultrasound waves can be generated without the transducer physically or directly contacting the skin. Nanoparticles 34, which can be introduced transdermally (e.g., via a lotion), or injected into the desired location, upon irradiation with ultrasonic waves 30, produce a cavitation zone 32 which damage the root of the hair follicle 28 by causing strong hydrodynamic flows. Such damage can prevent hair follicle 28 from re-generating, thereby resulting in a more permanent hair removal process that is non-invasive and non-scarring.

In a related aspect of the present disclosure, the methods of the present invention can be used to promote hair follicle development and stimulate hair growth. In accordance with this aspect of the application, application of nano- or microparticles to a section of a patients skin, and irradiating that section of the skin with ultrasonic energy waves, can result in the enhancement of hair growth by the stimulation of hair follicles. In a further, related aspect, such follicle stimulation may be by the stimulation of stem cells in the hair follicle, resulting in the promotion of hair follicle development.

B. Skin Rejuvenation

Ultrasound can be used for skin rejuvenation, due to the fact that ultrasound can penetrate in deep layers of skin and subcutaneous fat and produce thermal or mechanical damage to the tissues. Healing of the tissues irradiated with ultrasound results in regeneration (rejuvenation) of skin. To produce thermal damage, higher frequencies can be used, while lower frequency ultrasound can be used for mechanical (cavitation) damage to the tissues. The ultrasound-based skin rejuvenation can be performed with nano- or microparticles. Irradiation of the nano- or microparticles with ultrasound can result in damage to the skin tissues followed by rejuvenation. The mechanisms of damage include, but are not limited to, cavitation, thermal damage, and/or acoustic streaming. Ultrasound cavitation can be dramatically enhanced when nano- or microparticles are used because they serve as cavitation nuclei. The use of nano- or microparticles can substantially lower the cavitation threshold and increase cavitation activity. Collapsing cavitation bubbles can produce strong local hydrodynamic flows that damage the skin tissues. As an example, the size of the nano- or microparticles can be in the range from about 1 nanometer to about 200 microns and have different shape (spheres, cylinders, rods, shells, etc.). The nano- and microparticles include, but are not limited to gas particles (bubbles), liquid particles, solid (polymer, metal, dielectric, semiconductor) particles, or their combination or composites. Preferably, they contain a gas that substantially lowers the cavitation threshold and increases cavitation activity. Typically, the nanoparticles or microparticles used in accordance with the present invention are biodegradable, and include nano- or microbubbles, and porous solid (polymer, etc.) particles. The nano- or microbubbles suitable for use herein include, but are not limited to commercially available ultrasound contrast agents, such as ALBUNEX® (Molecular BioSystems, Inc., San Diego, Calif.), OPTISON™ (perflutren protein-Type A microspheres, GE Healthcare, Calfont St. Giles, United Kingdom), DEFINITY® (perflutren lipid microspheres, DuPont Pharmaceuticals), LEVOVIST® (galactose-palmitic acid, Berlex Canada, Pointe-Claire, Quebec), and bioSphere™ (Point Biomedical, San Carlos, Calif.). The porous (gas filled) polymer nano- or microparticles suitable for use with the present invention include, but are not limited to, PLA (polylactic acid), PGA (polyglycolic acid) and PLGA (polylactic-co-glycolic acid) particles. Solid (porous and non-porous) particles can produce more stable cavitation sufficient to damage the tissues compared to that produced by gas particles.

Ultrasound-based skin rejuvenation can be performed at low levels of ultrasound including diagnostic, close to diagnostic, or physiotherapeutic levels of ultrasound. The frequency of ultrasound is from about 20 kHz to about 900 MHz. Two or more frequencies can be used for more efficient skin rejuvenation. The ultrasound frequency (as well as other ultrasound parameters such as duration of irradiation, duty cycle, power, intensity, and size of the irradiation spot) can be selected to provide the optimal treatment effect with minimal damage to other tissues. In accordance with this and other aspects of the present disclosure, as indicated above, the frequency of the ultrasound irradiation suitable for use with the therapeutic methods described herein can range from about 20 kHz to about 900 MHz, more preferably from about 20 kHz to about 5 MHz, more preferably from about 20 kHz to about 1 MHz, and more preferably from about 20 kHz to about 500 kHz, including ranges and values between these ranges, such as between about 21 kHz and about 100 kHz, and between about 55 kHz and about 95 kHz.

One can use focused or non-focused ultrasound for skin rejuvenation. The focused ultrasound can be directed precisely to the area that should be damaged. Spherical or cylindrical focusing can be performed with one, two, or more focusing elements to provide multiple treatment spots with desired size and shape that may improve outcome and decrease treatment time. Masks made of ultrasound absorbing and/or reflecting materials can be used with focused or non-focused ultrasound for more efficient treatment. These masks can have holes with different size and shape and will provide damage to multiple areas with desired size and shape that may improve outcome and decrease treatment time. Combination of masks and focusing elements can also be used to achieve the best treatment effect.

The methods of the present invention can be applied to various, controlled depths below the surface of the skin, depending upon (among other things) the frequency of the ultrasonic waves. Consequently, and in accordance with the present invention, the methods of ultrasonic treatment for dermatological applications can be used to at subdermal depths ranging from about 0.001 mm to about 10 mm, as well as any value within this range, including about 0.01 mm, about 0.05 mm, about 0.1 mm, about 0.2 mm, about 0.3 mm, about 0.4 mm, about 0.5 mm, about 0.6 mm, about 0.7 mm, about 0.8 mm, about 0.9 mm, about 1.0 mm, about 2.0 mm, about 3.0 mm, about 4.0 mm, about 5.0 mm, about 6.0 mm, about 7.0 mm, about 8.0 mm, about 9.0 mm, and about 10.0 mm, as well as ranges between any two of these values, such between about 1.5 mm and about 6.5 mm. Alternatively, the methods can be used within one or more particular “layers” of the skin, such as both the epidermis and the dermis. In the instance of skin rejuvenation, the methods can extend well into the subcutaneous tissue, and even into the muscle and tendons if so required.

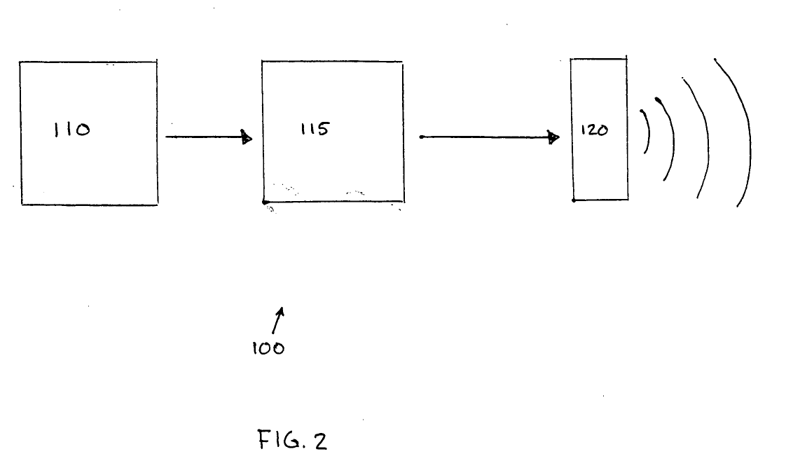

An ultrasound signal apparatus suitable for use within the present invention is illustrated in FIG. 2. As shown in FIG. 2, a signal generator 100 may comprise a frequency synthesizer module 110 producing an oscillatory output at a frequency suitable to excite the ultrasonic transducer 120. The oscillatory signal is then amplified by an amplifier and gate 115 to a voltage needed to excite the ultrasonic transducer 120. Finally, the excitation signal may be switched off by the gating circuit of the amplifier and gate 115. The frequency synthesizer 110 and the amplifier and gate 115 are programmable and can be controlled by an embedded microcontroller (not shown) or other programmable means. Thus the ultrasonic transducer 120 can be excited in a manner so as to force a more uniform coverage of tissue with ultrasound. Also, each transducer may be excited at a slightly different frequency to produce an interference pattern where the pressure peaks sweep in and out along the beam. The two embodiments may be combined to produce a beam that sweeps through the targeted tissue while the pressure peaks sweep along the beam. While the present invention contemplates the use of multiple transducers, a single transducer can also be used with a signal generator generating a plurality of acoustic signals having different frequencies, phases and amplitudes. A signal generator may also be used to generate a plurality of acoustic signals having randomly generated frequencies, phases and amplitudes. The signal generator may also use a white noise source to generate the acoustic signals.

Ultrasound is absorbed by tissues, and at high power causes a temperature increase within the tissue. The amplitude of the excitation voltage can be manipulated to reduce the heating effect. By using the gating circuit of the amplifier and gate 115 (FIG. 2), short bursts of ultrasound may be produced so that the average power delivered to the targeted tissue may be reduced while the intensity of the ultrasound may be kept relatively high during the short ultrasound burst. Also, as noted above, certain embodiments of the invention may use phased arrays of transducers to move an ultrasound beam around the targeted tissue. Likewise, an array employing varying frequencies can be used to produce interference patterns of traveling waves of ultrasound that move through the targeted tissue.

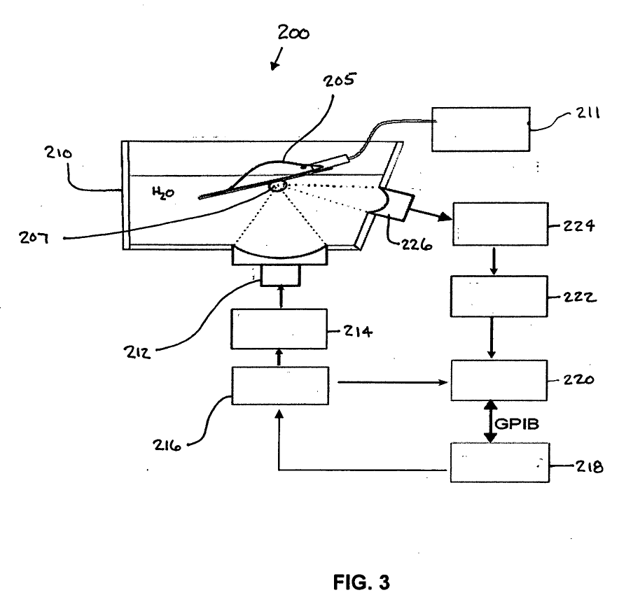

Turning now to FIG. 3, yet another ultrasound system for use in accordance with the present disclosure is described, wherein the system 200 comprises a confocal configuration, meaning that the irradiating and detecting transducers are focused in one spot, ultrasonic irradiation focal point 207. This system allows for the measurement of cavitation signals with minimal noise associated with other, non-linear acoustic effects. As shown in the figure, a patient 205, in this case a mouse (such as tested in the Examples, described in further detail below), is located on an optional support plane or platform which is at least partially submerged in a treatment tank 210 containing water at about 37° C. The patient 205 is optionally connected to an anesthetic source, 211, which may be any appropriate anesthetic, such as isoflurane, a halogenated, volatile anesthetic which induces and maintains general anesthesia by depression of the central nervous system. The tank 210 is fitted with a focused transducer, 212, and a confocal detector, 226. The transducer 212 is a focused ultrasound generator having a resonance frequency ranging from about 1 MHz to about 5 MHz, and irradiates focused ultrasound waves which are directed to the skin or subcutaneous tissue of the patient. The detector 226 has a resonance frequency of about 5 MHz, for detection of cavitation signals and activity at 5 MHz. Those of skill in the art will recognize that the power of the transducer and detector may be of any appropriate resonance frequency, and are not limited to those powers given in this example. The system also comprises an Rf power amplifier 214, such as a 2100 L ENI amplifier, a signal or pulse/function generator 216, such as an 8116A generator (available from Hewlett-Packard), a highpass filter 224 associated with the confocal detector, a signal amplifier 222, an ADC board 220, and a computer or other suitable, human-machine interface (HMI) 218.

With continued reference to FIG. 3, cavitation is induced in the patient 205 using short (30 ms) ultrasonic pulses from transducer 212, with a repetition rate of about 20 Hz. When cavitation is induced in the tissues at focal point 207 by the focused ultrasound, an acoustic (ultrasound) wave (also known as a cavitation wave) is generated in the focus because the cavitation bubbles collapse and produce local hydrodynamic flows. This cavitation wave propagates in all directions. The 5-MHz transducer/confocal detector 226 is a focused ultrasound detector which is focused in the focal point of the ultrasound-generating transducer 212, thus forming a confocal irradiation-detection system. This confocal detection system detects a part of the cavitation wave that propagates in its direction. The cavitation signal is then filtered by the high pass filter 224 in order to remove any unwanted noise, is amplified by the signal amplifier 222, digitized by the analog-to-digital converter (ADC) 220, recorded by the scope, and finally analyzed by the computer 218.

C. Therapeutic Agents

In accordance with further aspects of the present disclosure, the ultrasound-based therapeutic treatment methods described herein, especially those methods comprising the use of ultrasonic irradiation and nano- or microparticles enhance the cavitation, may further optionally comprise one or more therapeutic agents, the selection of which will depend upon the specific application of the present methods. Consequently, in accordance with these aspects of the present disclosure, the methods of treating dermatologically-related disorders or associated problems may further comprise preparing a step or series of steps of preparing a dermatologically therapeutic composition, the dermatologically therapeutic composition comprising one or more types of nano- or microparticles, one or more therapeutic agents, and optionally a carrier or other formulation excipient, as necessary. For example, for the use of the present methods in skin rejuvenation, such as wrinkle removal, the methods may also comprise the inclusion of one or more rejuvenating agents with the nano- or microparticles, while for hair growth, the use of one or more hair growth promoting agents may be desired in combination with the nano- or microparticles. While many therapeutically useful drugs cannot be delivered transdermally because of their low ability or inability to substantially penetrate the dermis, it is believed that the combination of the nano- or microparticles with the ultrasound irradiation methods and techniques described herein can be used to deliver drugs into the skin, especially the subcutaneous tissues. In accordance with this aspect of the present disclosure, methods of therapeutic dermatological treatments include irradiating a composition comprising one or more nano- or microparticles and a dermatologically therapeutic agent with ultrasonic radiation for a period of time, and for an appropriate number of applications, that the therapeutic agent may penetrate into the subcutaneous layer of the dermis. In further accordance with this aspect, the irradiation may allow a drug or combination of drugs to penetrate into the stem cells of hair follicles so as to alter or kill the hair follicles, thereby resulting in hair removal, especially permanent hair removal.

Consequently, in accordance with the methods of the present disclosure, the methods comprising the use of nano- or microparticles may further comprise the preparation and use of one or more dermatologically therapeutic agents in combination with the nano- or microparticles, such therapeutic agents including but not limited to rejuvenating agents, hair removal/hair growth inhibiting agents, hair growth promoting agents, skin cancer therapeutic drugs (including 5-fluorouracil (5-FU), vinblastine, stilbenes and stilbene derivatives, TAXOL®, carmustine, BCNU (topical), antiangiogenesis agents, and combinations thereof), anesthetics, anti-inflammatory agents, antibacterial agents, antiviral agents, keratin softening agents, dermal moisturizing agents, as well as mixtures and combinations of such therapeutic agents.

Suitable rejuvenation agents for use with the present invention include but are not limited to retinoids; vitamins, such as vitamin B₁₂, Vitamin C, and vitamin E; AHA'S; BHA's; keratolytics; sunscreens; and combinations thereof. Retinoids include but are not limited to vitamin A (retinol, ROL) and its metabolites, such as retinaldehyde (RAL), all-trans-retinoic acid (ATRA), and 13-cis-retinoic acid, synthetic analogs of retinol such as etretinate and acitretin, poly-aromatic analogues and arotinoids of retinol such as adapalene, and tretinoin, as well as the solvates, salts, polymorphs, and prodrugs of these compounds (such as retinyl palmitate), and combinations thereof. Vitamins suitable for use as dermatological therapeutic agents in accordance with the present disclosure include but are not limited to vitamin E (tocopherol), ascorbic acid (vitamin C), tocopherol acetate, tocopherol sorbate, vitamin D, coenzyme Q10, and combinations thereof. Alpha hydroxy acids (AHAs) suitable for use herein include any of the group of hydrophilic organic carboxylic acids having a hydroxy group in the alpha position, including but not limited to glycolic acid, lactic acid, citric acid, pyruvic acid, malic acid, tartaric acid, and combinations thereof. Beta hydroxy acids (BHAs) suitable for use herein include but are not limited to salicylic acid and related compounds, as well as those structurally similar compounds capable of inducing epidermal proliferation and activation of factor XIII and/or TNF-alpha.

Hair growth inhibitors suitable for use as therapeutic agents with the methods and systems of the present invention include but are not limited to antiandrogens, such as flluridil, RU58841 [Battmann, T., et al., J. Steroid Biochem. Mol. Biol., Vol. 48(1): pp. 55-60 (1994)], 17 alpha-Propylmesterolon, inocoterone, CASODEX® and NILANDRON®; androgen inhibitors; chromaphores; photosensitizers; enzymes, such as elastase-like enzymes; matrix metalloproteinases; mercaptopropionamides and their derivatives; phosphonic acid derivatives; and combinations thereof.

Therapeutic agents which may be included in the methods and systems of the present disclosure for the purpose of retarding hair loss and/or promoting hair growth include, but are not limited to, inhibitors of 5-alpha reductase; thymosin beta-4 (Tβ₄; a 43 amino acid polypeptide that interacts with and sequesters G-actin) [see, Philp, D., et al., Mechanisms of Ageing and Development, Vol. 125: pp. 113-115 (2004); Malinda, K. M., et al., FASEB J., Vol. 11: pp. 474-481 (1997)]; cyclosporine and cyclosporine derivatives, such as cyclosporines A-Z, thiocyclosporins, as well as analogs, salts, prodrugs, and polymorphs thereof; rapamycin; FK506; sarcosine, thiosarcosine, and their derivatives, analogs, and prodrugs; dilators of peripheral blood vessels; pyrollidine derivatives and pyrrolidine carboxylate compounds; thyroxanes; pyrimidines and pyrimidine-3-oxides; compounds with an affinity for FKBP-type immunophilins; and combinations of such agents.

The compositions of some embodiments of the present invention may contain, in addition to one or more therapeutic agents and micro- or nanoparticles, additional, optional ingredients such as carrier, solvent, excipient or vehicle ingredients. These may include, by way of example and not by way of limitation, water, acetone, ethanol, ethylene glycol, propylene glycol, butane-1,3-diol, acrylates copolymers, isopropyl myristate, isopropyl palmitate, mineral oil, butter(s), aloe, talc, botanical oils, botanical juices, botanical extracts, botanical powders, other botanical derivatives, lanolin, urea, petroleum preparations, tar preparations, plant or animal fats, plant or animal oils, soaps, triglycerides, and keratin(s). Mixtures formed by the combination of the above ingredients to form soaps, lotions, tinctures, creams, pastes, emulsions, gels/jellies, aerosols, sprays or ointments which are non-toxic and pharmaceutically, medically, dermatologically, and/or cosmetically acceptable may also be comprised within embodiments of the present invention.

Additionally, moisturizers, sunscreens, fragrances, dyes, thickening agents such as paraffin, jojoba, paba, and waxes, surfactants, humectants, occlusives, hygroscopic agents, emulsifiers, emollients, lipid-free cleansers, antioxidants and lipophilic agents, maybe added to formulations of the present compositions if desired.

As indicated above, in addition to these and other vehicles, it shall be understood that the therapeutic, dermatological, pharmaceutical, medical, and/or cosmetic compositions of the present invention may optionally include other therapeutic ingredients such as those that may improve or eradicate itching, irritation, pain, inflammation, age spots, keratoses, wrinkles, and other blemishes or lesions of the skin. By way of example and not by way of limitation, analgesics, anesthetics, antiacne agents, antibacterial agents, anti-yeast agents, anti-fungal agents, antiviral agents, antibiotic agents, porbiotic agents, anti-protozal agents, anti-pruritic agents, antidandruff agents, anti-dermatitis agents, anti-emetics, anti-inflammatory agents, anti-hyperkeratolyic agents, anti-dry skin agents, antiperspirants, anti-psoriatic agents, anti-seborrheic agents, hair conditioners, hair treatments, anti-aging agents, anti-wrinkle agents, antihistamine agents, disinfectants, skin lightning agents, depigmenting agents, vitamins and vitamin derivatives, gamma-linolenic acid (GLA), beta carotene, quercetin, asapalene, melaluca alternifolia, dimethicone, neomycin, corticosteroids, tanning agents, zinc/zinc oxides, sulfur agents, hormones, retinoids, clotrimazole, ketoconazole, miconazole, griseofulvin, hydroxyzine, diphenhydramine, pramoxine, lidocaine, procaine, mepivacaine, monobenzone, erythidocaine, erythromycin, tetracycline, clindamycin, meclocline, hydroquinone, minocycline, naproxen, ibuprofen, theophylline, cromolyn, alburterol, retinoic acid and its derivatives, hydrocortisone and its derivatives, mornetasone, desonide, trimcinolone, predisolone, NUTRACORT™ brand topical steroid application, salicylic acid, phospholipids, calamine, allantoin, isohexadelane, ceresin, galcipotriene, DOVONEX™ brand dermatological preparation, anthralin, betamethasone valerate, betamethasone diproprionate, trimcinolone acetonide, fluocinonide, clobetasol propionate, benzoyl peroxide, crotamition, propranolon, promethanzine, and mixtures or derivatives thereof may be added to embodiments of the present invention to improve or alter their effectiveness.

The compounds used in the formulations and therapeutic compositions of the present invention may be used as their therapeutic, dermatological, pharmaceutical, medical, and/or cosmetic acceptable salts, solvates, prodrugs, hydrates, or polymorphs. Salts may be prepared from pharmaceutically and chemically acceptable non-toxic acids or bases including inorganic and organic acids and inorganic and organic bases. Such salts may contain, by way of example and not by way of limitation, the following ions: Acetate, benzensulfonate, benzoate, camphorsulfonate, citrate, fumarate, gluconate, hydrobromide, hydrochloride, lactate, maleate, mandelate, mucate, nitrate, pamoate, phosphate, succinate, sulfate, tartate, pyruvate and the like. Such salts may also contain the following cations: aluminum, calcium, lithium, magnesium, potassium, sodium, zinc, benzathine, chloroprocaine, choline, diethanolamine, ethylenediamine, meglumine and procaine.

While most of the examples, systems and methods described so far herein seem most applicable to a clinical setting, it is important to note that the disclosed treatment methods and systems are envisioned to be suitable for use not only in clinics, but also in cosmetic salons (in the case of unwanted hair removal, for example), and for home use. In particular, for hair removal, an smaller, ultrasound-based home device may be developed (something like home ultrasound epilator), while for stimulation of hair growth one can use ultrasound brushes or similar home devices. Same is true for skin rejuvenation: home device for skin rejuvenation.

The following examples are included to demonstrate preferred embodiments of the invention. It should be appreciated by those of skill in the art that the techniques disclosed in the examples which follow represent techniques discovered by the inventors to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the scope of the invention.

### EXAMPLES

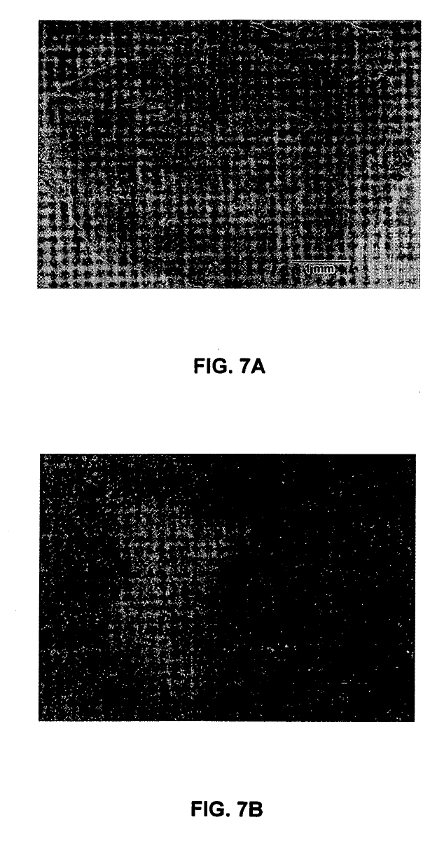

Microscope images illustrated in FIGS. 7A-10B were obtained using standard H&E staining techniques and an Olympus bright field optical microscope (Olympus Optical Co., Japan), with digital video cameras used to detect damage in the tissues. These staining and optical microscopy techniques are well known to those in the art and are widely used for the detection of damage in tissues.

### Example 1

### Cavitation Studies

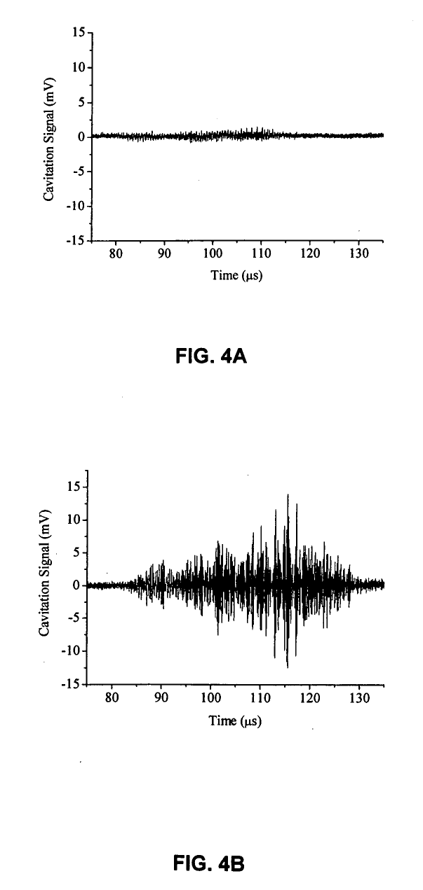

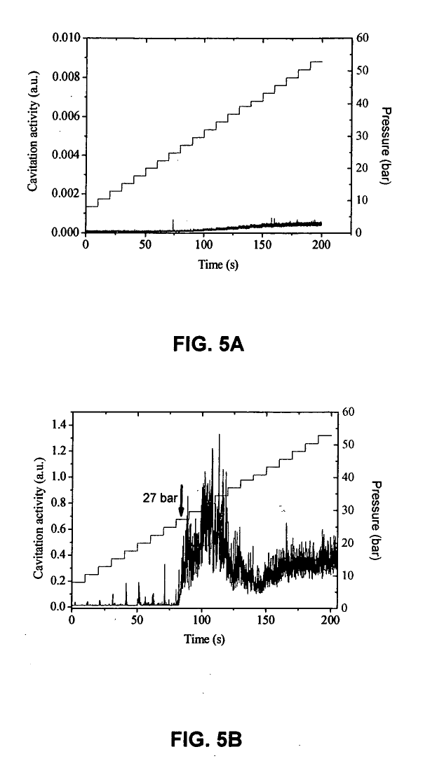

A study on the cavitation threshold and activity in pure water and in water containing poly(lactic-co-glycolic acid) (PLGA) nanoparticles as well as in vivo in nude mice. FIGS. 4A and 4B illustrate the cavitation signals obtained at the same ultrasound pressure from pure water (4A) and from containing PLGA nanoparticles (4B), respectively. As is readily apparent from these figures, the cavitation signal obtained from water with PLGA nanoparticles was significantly greater. The signals were integrated using a procedure described in detail in the article by Larina, I. V., et al., [Technol Cancer Res. Treat., Vol. 4(2): pp. 217-226 (2005)] to measure cavitation activity at different pressure (cavitation activity is proportional to concentration of cavitation bubbles and strength of individual cavitation events). In further tests, and as illustrated in FIGS. 5A and 5B, cavitation activity was measured in both pure water (FIG. 5A) and in water containing PLGA nanoparticles (FIG. 5B), respectively, over time with an increase in ultrasound pressure. As illustrated, the ultrasound pressure (right Y-axis) was increased in a step-by-step manner over the course of the measurements. As can be seen by comparing the two figures, the cavitation activity is substantially greater in water containing PLGA nanoparticles than that measured in pure water.

These experiments allowed for the determination of a cavitation threshold (a sharp increase of cavitation activity), which as shown in FIG. 5B was about 27 bar for water with PLGA nanoparticles at these experimental conditions. In contrast, no sharp increase of cavitation activity was detected in pure water, likely because the cavitation threshold was very high. These data illustrated in FIGS. 5A and 5B demonstrate that: (1) cavitation threshold and activity can be measured with a feed-back system, in accordance with the systems of the present disclosure, and (2) PLGA nanoparticles substantially decrease cavitation threshold of a solution while simultaneously increasing the cavitation activity of a solution.

### Example 2

### In Vivo Studies

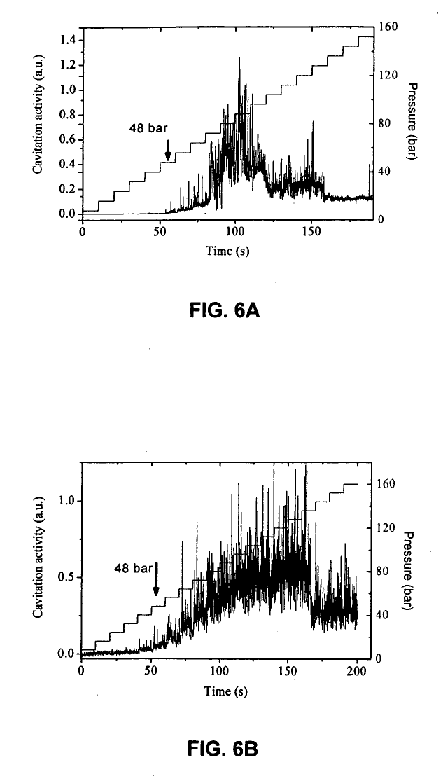

In the in vivo experiments, Optison™ (Amersham, Mallinkrodt) or PLGA nanoparticles were injected into the tail vein of nude mice and the cavitation signals and activity were measured using the same approach as used in the experiments with water, described above. FIG. 6A shows cavitation activity measured from the subcutaneous tissue of a mouse injected with Optison™ prior to irradiation. As can be seen from the photomicrograph, cavitation activity can be detected with a threshold of about 48 bar.

However, the cavitation activity decreased rapidly due to degradation of Optison™ upon ultrasound irradiation. Injection of another mouse with PLGA nanoparticles also induced cavitation activity in the irradiated tumor (with almost same threshold), as shown in FIG. 6B. However, the PLGA nanoparticles produce stable cavitation for a longer time because they degrade at a much slower rate. These studies demonstrate that PLGA nanoparticles: (1) substantially lower cavitation threshold; (2) produce cavitation in vivo; (3) produce more stable cavitation in vivo compared with that obtained with Optison™; and (4) the feed-back system is capable of detecting cavitation in vivo and can be used for efficient and safe treatment of skin or subcutaneuous tissues.

### Example 3

### Ultrasound-Induced Tissue Damage Studies

In addition to the benefits of can induce precise damage in multiple areas of tissue at a desired depth using such a system. FIG. 7A shows ultrasound-induced damage in multiple areas at different depth from the skin surface and lateral locations. The single areas of damage have millimeter or submillimeter size. FIG. 7B shows a damaged area with a submillimeter width and one-millimeter length. This size and depth of the damage areas is desirable for treatment of skin and/or subcutaneous tissues (e.g. skin rejuvenation) and cannot be achieved at depths greater than 0.5 mm with standard laser- (or in general, light-) based techniques/systems. The ultrasound beam can be moved laterally over the skin surface to produce desirable pattern of damaged areas at the desired, subcutaneous depths.

### Example 4

### Non-Focused Ultrasound Combined with Topical Application of Micro- or Nanoparticles for Skin Rejuvenation

One can use non-focused ultrasound systems in combination with micro- or nanoparticles to produce desirable damage to skin or subcutaneous tissues. Irradiation can be performed at different ultrasound parameters depending on type of treatment procedure, type of the tissue to be treated, size and depth of desirable damage, intensity of desirable damage, as well as material and size the of micro- or nanoparticles used for the procedure. As an example, one can use lipid-based microparticles (such as Definity®, a perflutren lipid microsphere commercially available from Bristol Myers Medical Imaging) or non-lipid based micro-particle (such as Optison™) and non-focused ultrasound with 1-MHz frequency, 7-bar pressure amplitude, 100-microsecond pulse duration, 200-Hz repetition rate, and 100-second irradiation (treatment) time for a treatment area of 3 sq. cm. These parameters were used in in vivo studies in pigs not for limitation, but for demonstration purposes only. It should be noted that without micro- or nanoparticles ultrasound with similar parameters cannot produce any tissue damage and is being widely and safely used in ultrasonic imaging (including Doppler applications) as well as in physiotherapy.

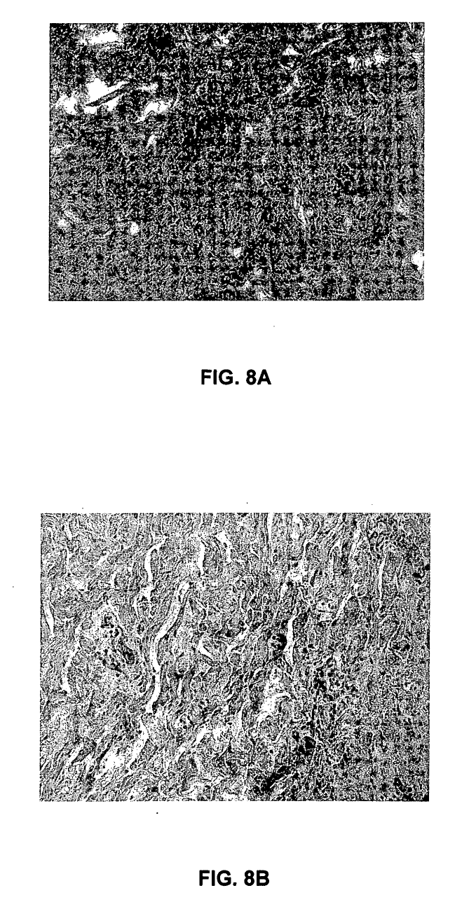

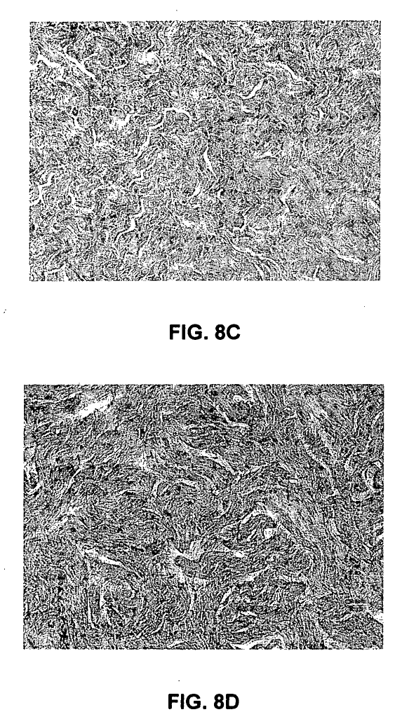

FIGS. 8A-8C illustrate the dermal collagen of a pig in three different treatment areas: ultrasound+Definity® (lipid-based microparticles), ultrasound+Optison™ (Albumin-based microparticles), and ultrasound+water, respectively. The treatment areas were irradiated in vivo and then analyzed with H&E staining. Combination of ultrasound with Definity® induced pronounced damage to dermal collagen, while ultrasound irradiation with Optison™ produced only minor damage. Treatment by ultrasound with water produced no damage and histology of this treatment area was similar to that of control (non-irradiated) areas, as illustrated in FIG. 8D.

Definity®, Optison™, and water were rubbed on the skin surface for a few minutes prior to irradiation, in order to increase penetration of the solution into the dermis. Since Definity® and other lipid-based micro- or nanoparticles penetrate into the dermis much better than non-lipid based micro- or nanoparticles, they can be used to achieve best treatment effects. Therefore, preferably, lipid-based micro- or nanoparticles may be used for treatments (such as skin rejuvenation, etc.) and rubbed prior to ultrasonication. To optimize treatment effects (best rejuvenation and rapid growth of new collagen and healing after the procedure) one can irradiate multiple areas with predetermined locations, size, and depth of the damaged areas as well as with predetermined intensity of the damage.

### Example 5

### Hair Removal

A similar approach as described in Example 4 above can be used for hair removal.

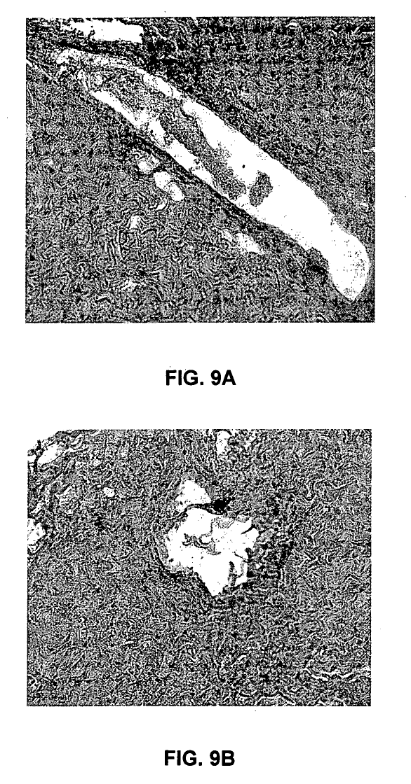

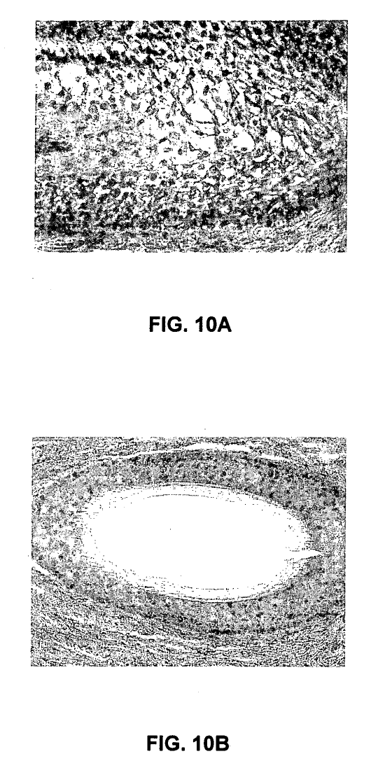

The same microparticles and irradiation parameters were used to produce damage to hair follicles. FIGS. 9A and 9B show damage induced in hair follicles by non-focused ultrasound in combination with Optison™ which was applied topically to the skin surface prior to ultrasound irradiation. The tissue was cut along (FIG. 9A) or across (FIG. 9B) the hair follicles. The area was mechanically epilated prior to application of Optison™. This allowed for penetration of Optison™ into the hair follicles. Application of Definity® resulted in less damage to hair follicles under the same experimental conditions (FIG. 10A). This is likely due to the fact that Optison™ exhibits a higher cavitation activity compared to that of Definity®, while penetration of these microparticles into the hair follicle may be the same due to the epilation. FIG. 10B shows hair and hair follicle irradiated by non-focused ultrasound in combination with water which was applied (for acoustic contact) topically to the skin surface prior to ultrasound irradiation. No damage was induced to the hair or to the hair follicle. This hair and follicle structure is typical of that for intact hair and hair follicle. This study demonstrates that: (1) ultrasound in combination with micro- or nanoparticles can be used for hair removal; (2) one can use the ultrasound-based hair removal in combination with other hair removal techniques; (3) microparticles with higher cavitation activity (such as Optison™) produce best treatment effects; and (4) effective hair removal can be performed at low levels of ultrasound typical for diagnostic and physiotherapeutic ultrasound systems.

### Prophetic Example 1

### Wrinkle Reduction

A photo-aged female subject is tested for skin elasticity and visible skin wrinkles before treatment. Albunex® (air-filled albumin microspheres suspended in a solution of 5% (w/v) human albumin, wherein the suspension is sterile, non-pyrogenic and isotonic, with a pH of 7.0 and a viscosity of 1.4 relative to water), or, optionally and equally acceptable, Definity® lipid-based microparticles, is then applied to the target area of the patient, and the area is then subjected to ultrasound treatment at a frequency of about 500 MHz for a period of time. The procedure is repeated over 12 weeks to the entire facial area. The average reduction in visible wrinkles is observed every 4 weeks and recorded.

The various steps described or claimed herein can be combined with other steps, can occur in a variety of sequences unless otherwise specifically limited, various steps can be interlineated with the stated steps, and the stated steps can be split into multiple steps. Unless the context requires otherwise, the word “comprise” or variations such as “comprises” or “comprising”, should be understood to imply the inclusion of at least the stated element or step or group of elements or steps or equivalents thereof, and not the exclusion of any other element or step or group of elements or steps or equivalents thereof. Also, any directions such as “top,” “bottom,” “left,” “right,” “upper,” “lower,” and other directions and orientations are described herein for clarity in reference to the figures and are not to be limiting of the actual device or system or use of the device or system. The device or system may be used in a number of directions and orientations.

The invention has been described in the context of preferred and other embodiments and not every embodiment of the invention has been described. Obvious modifications and alterations to the described embodiments are available to those of ordinary skill in the art. The disclosed and undisclosed embodiments are not intended to limit or restrict the scope or applicability of the invention conceived of by the Applicants, but rather, in conformity with the patent laws, Applicants intends to protect all such modifications and improvements to the full extent that such falls within the scope or range of equivalent of the following claims.

What is claimed is:

1. A method for the dermatological treatment of a region of skin or subcutaneous tissue, the method comprising:
irradiating the skin with ultrasonic radiation having a frequency from about 20 kHz to about 4 GHz for a period of time sufficient to therapeutically effect the region of skin or subcutaneous tissue.

2. The method of claim 1, further comprising applying a therapeutically effective amount of nanoparticles or microparticles to the region of skin.

3. The method of claim 2, wherein the microparticles or nanoparticles are applied subcutaneously to the region of skin.

4. The method of claim 2, wherein the microparticles or nanoparticles are applied topically to the region of skin.

5. The method of claim 2, wherein the microparticles or nanoparticles are lipid-based.

6. The method of claim 2, wherein the microparticles or nanoparticles are not lipid-based.

7. The method of claim 1, wherein the ultrasonic irradiation is administered at a pressure ranging from about 1 bar to about 200 bar.

8. A method for removing one or more hair follicles from a region of skin on a mammal, the method comprising:
applying a therapeutically effective amount of nanoparticles or microparticles to the region of skin; and
irradiating the surface of the skin with ultrasonic radiation for a period of time,
wherein the ultrasonic radiation disrupts the one or more hair follicles in the region being treated.

9. The method of claim 7, further comprising applying one or more therapeutic agents capable of causing hair follicle growth inhibition to the region of skin.

10. The method of claim 9, wherein the one or more therapeutic agents are selected from the group consisting of antiandrogens, androgen inhibitors, chromaphores, photosensitizers, enzymes, phosphonic acid derivatives, matrix metalloproteinases, and combinations thereof.

11. The method of claim 9, wherein the one or more therapeutic agents are applied in combination with the nanoparticles or microparticles.

12. A method for enhancing growth of one or more hair follicles from a region of skin on a mammal, the method comprising:
applying microparticles or nanoparticles to the region of skin; and
irradiating the region of skin with ultrasonic radiation for a period of time sufficient to stimulate stem cells within the hair follicle,
wherein the irradiation step is performed a number of times ranging from one time to more than 100 times over the period of time.

13. The method of claim 12, further comprising applying one or more therapeutic agents capable of stimulating hair follicle growth or retarding hair follicle loss.

14. The method of claim 13, wherein the one or more therapeutic agents are selected from the group consisting of cyclosporins, thyroxanes, pyrimidines, pyrimidine-3-oxides, inhibitors of 5-alpha reductase, thymosin beta-4, pyrrolidines, and combinations thereof.

15. The method of claim 13, wherein the one or more therapeutic agents are applied in combination with the nanoparticles or microparticles.
